(19) United States
(12) Patent Application Publication
(10) Pub. No.: US 2010/0233675 A1
(43) Pub. Date: Sep. 16, 2010

(54) ANALYTE MANIPULATION AND DETECTION

(75) Inventors: Denise Barrault, Midloth (GB); Stuart Polwart, Midloth (GB); David Thomson, Midloth (GB); David Pritchard, Dundee (GB); Erling Sundrehagen, Scotland (NO)

Correspondence Address: KNOBBE MARTENS OLSON & BEAR LLP, 2040 MAIN STREET, FOURTEENTH FLOOR, IRVINE, CA, 92614, US

(73) Assignee: ITI SCOTLAND LIMITED, GLASGOW (GB)

(21) Appl. No.: 12/438,003

(22) PCT Filed: Aug. 17, 2007
(86) PCT No.: PCT/GB07/03142

§ 371 Date: Jul. 16, 2009

(30) Foreign Application Priority Data
Aug. 18, 2006 (GB) 0616508.8

(51) Int. Cl. C12Q 1/70; C07H 21/04; C07K 1/14; C12Q 1/68; C07H 21/02; C12M 1/34

(52) U.S. Cl. 435/5; 536/23.1; 530/412; 435/6; 435/287.2

(57) ABSTRACT

Provided is a method for separating two or more analytes in a fluid, which method comprises: (a) binding each different analyte to a different functional particle in one or more binding zones, to produce two or more bound analytes; (b) allowing the bound analytes to move through a separating conduit to two or more separate functional zones; wherein, each different functional particle has, or can be controlled to have, a different function in the fluid as compared with the other functional particles; and wherein the separating conduit separates into two or more functional conduits, such that the separating conduit serves to separate the bound analytes into the separate functional conduits by means of the different functions of the different functional particles. Also provided is an apparatus for separating two or more analytes in a fluid, which apparatus comprises: (a) a binding zone; (b) two or more functional conduits; (c) a separating conduit connecting the binding zone to the two or more functional conduits; (d) a transporter for transporting the analyte through the separating conduit from the binding zone to the two or more functional conduits; and (e) optionally one or more concentrating zones in connection with at least one of the functional conduits.

The present invention concerns methods for manipulating and detecting analytes, especially in microfluidic systems. The method relates in particular to methods for separating different analytes from the same sample. The invention is particularly advantageous, since its concentration aspects allow analytes to be detected without complicated conventional concentration and amplification techniques, whilst its separation aspects allow a plurality of different analytes in a single sample to be detected, or separately manipulated. In particular, it reduces the effects of the depletion layer experienced in microfluidic devices by providing a certain degree of active transport towards the detection zone.

It has been known to employ buoyant particles, and other types of particle (such as magnetic particles and high density particles) in methods of analysis, in particular in biological assays. In addition to this, methods using buoyant beads to remove waste from the surface of large volumes of water, such as swimming pools, are also well-known. Typically, hollow particles that are buoyant and are capable of attaching to bacterial contaminants in the water via an antibody linked to the surface of the particle are mixed with the water and upon rising to the surface, the bacteria and particle mixture is ‘skimmed’ from the surface to detect the pool contaminants. This has been carried out for cryptosporidium detection in swimming pools.

Methods for detecting analytes using solid particles, in particular magnetic and latex beads, have been around for some time. For example, common assay methods use magnetic beads which are added to the sample to be assayed. The beads carry a ligand on their surface which enables it to bind specifically to a target analyte. A magnetic field is then applied, enabling the beads and the bound material to be separated from the rest of the sample. In many cases the analyte is then measured by detection of a fluorescence-based emission, and can be used in conjunction with flow cytometric analysis. Such methods have been used for in vitro diagnostics against desired targets such as cells, nucleic acids, proteins and other types of biomolecule.

However, these types of existing methods do not concentrate the analytes into a specific area within a channel, making it difficult to detect them using a chip- or microarray-based method on a planar surface. Also, mixing particles with different specificities to the analytes in solution is not possible, as it would be impossible to distinguish between them, once bound between the different pairings. Furthermore, these methods do not reduce the problem of the analyte depletion layer, which is created during the binding step in microfluidic devices that use surface bound transducers and detectors. This endemic problem limits the inherent sensitivity and increases the time to result of assays and tests carried out in microfluidic devices.

It is an aim of the present invention to solve the problems associated with known techniques, including those described above. It is a further aim of the invention to develop improved methods for processing analytes (such as concentrating, actively transporting, and separating) and detecting analytes.

Accordingly, the present invention provides a method for separating two or more analytes in a fluid, which method comprises:
(a) binding each different analyte to a different functional particle in one or more binding zones, to produce two or more bound analytes;
(b) allowing the bound analytes to move through a separating conduit to two or more separate functional zones;
   wherein, each different functional particle has, or can be controlled to have, a different function in the fluid as compared with the other functional particles; and wherein the separating conduit separates into two or more functional conduits, such that the separating conduit serves to separate the bound analytes into the separate functional conduits by means of the different functions of the different functional particles.

In all embodiments of the present invention, it is particularly preferred that the separating conduit is a microfluidic separating conduit and the functional conduits are microfluidic functional conduits.

In a typical embodiment, the functional particle is attached to a recognition agent that is specific for the analyte. Generally, the fluid may be any suitable fluid. Preferably, the fluid is an aqueous fluid.

In a preferred embodiment of the method, after step (b), the bound analyte is transported to a concentrating zone near one or more detection elements in the fluid. This step is not essential, and in some embodiments, the detection element may be above or below the exit from the separating conduit, so that natural buoyant migration due to the buoyant particles, or natural negative-buoyancy migration due to highly dense particles, will be sufficient to both transport and concentrate the bound analytes to the detection element.

In some embodiments of the present method, the fluid contains a plurality of different analytes. In such embodiments it is preferred that a different recognition agent is provided for each different analyte, in order that each different analyte is attached to a particle having a different functionality. This enables the different analytes to be separated based upon the different functionality of the functional particles.

As mentioned above, preferably each particle employed in the methods of the present invention is attached to a recognition agent. In one embodiment, each particle may be attached to a single recognition agent. In this embodiment, the particles may all be attached to the same recognition agent (if only a single analyte is to be detected) or some particles may be attached to different recognition agents (if more than one analyte is to be detected). The number of different recognition agents will depend on the number of analytes in the sample that are under investigation. In alternative embodiments, each particle may be attached to more than one recognition agent. The recognition agents attached to a single particle may be the same (for example if it is desirable to increase the binding potential of the particle to the analyte, or to attach more than one analyte species to a single particle) or may be different (e.g. if it is desirable to attach any of the analytes under investigation to any of the particles). In some of the latter embodiments, all of the different types of recognition agent in the system may be attached to a single particle so that any or all of the possible analytes may bind to a single particle.

It is particularly preferred that the functional particles have, or can be controlled to have different buoyancies in the fluid. Such particles may comprise buoyant beads, dense beads, and/or magnetic beads with buoyancy controllable by a magnetic field, or having neutral buoyancy and being attracted to the magnetic field in a controlled manner. In these embodiments, a preferred method provided by the invention is a method for separating two or more analytes in a fluid, which method comprises:
(a) binding each different analyte to a different particle in a binding zone, to produce two or more bound analytes;
(b) allowing the bound analytes to move through a separating conduit to two or more separate functional zones;
   wherein each different particle is attached to a different recognition agent that is specific for a different analyte, and each different particle has, or can be controlled to have, a different buoyancy in the fluid as compared with the other particles; and wherein the separating conduit separates into two or more functional conduits, each functional conduit being situated at a different height from the other functional conduits, such that the separating conduit serves to separate the bound analytes into separate functional conduits by means of the different buoyancies of the different particles.

In the methods of the present invention the functional particles are not especially limited, provided that the function of one type of particle does not unduly impair the function of another type of particle. As mentioned above, the functional particles are preferably selected from:
(a) particles that are buoyant in the fluid;
(b) magnetic particles whose buoyancy can be controlled by the application of a magnetic field or whose buoyancy is neutral and whose attraction to the magnetic field can be controlled; and
(c) particles that are more dense than the fluid.

In the method of the invention, when the particles are buoyant they may rise toward the functional zone. When the particles are magnetic, they may be controlled to rise, fall or move laterally toward the desired functional zone. When the particles are dense, they may fall toward the functional zone. Similarly, the particles' functions may cause them to move toward their respective desired functional conduits.

The method of the present invention is advantageous because it allows a more rapid detection of analytes in a sample by reducing the number of processing steps conducted on the sample. It provides a method to separate and concentrate analytes in the vicinity of the detector, reducing the effect of the depletion layer experienced in microfluidic devices. By virtue of allowing multiplexing it reduces the number of experiments the user has to carry out and the number of instruments that have to operate to process the same sample for different tests. Further, it reduces the amount of laboratory equipment required, making the method easier, and less costly, to perform. The invention is particularly advantageous, since its concentration aspects allow analytes to be detected without complicated conventional concentration and amplification techniques, and provides active transport of the analytes to the detection zone, whilst its separation aspects allow a plurality of different analytes in a single sample to be detected, or separately manipulated.

The present invention will be described further by way of example only, with reference to the following Figures in which:

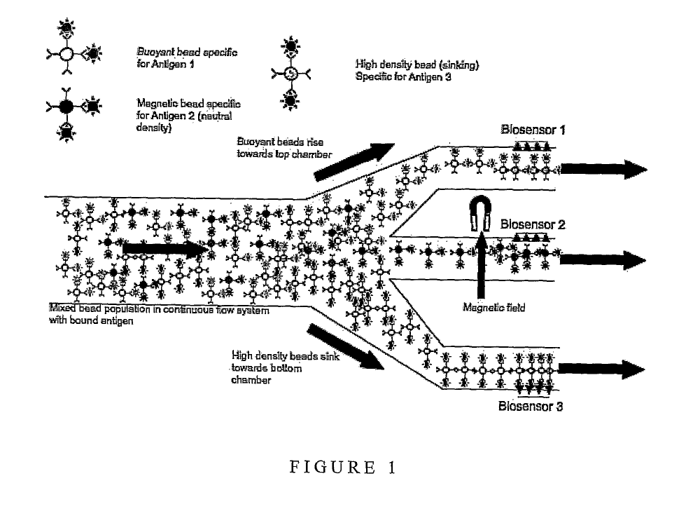

FIG. 1 shows as a schematic, an example of the layout of a separating apparatus in one embodiment of the present invention.

The present invention will now be described in more detail.

The methods of the present invention may be employed to detect any type of analyte, provided that it may be attached to the particles. However, it is preferred that the methods are performed using a fluid that comprises a sample containing the analyte. Typically, the sample comprises a crude lysate of solid tissue, a crude lysate of a cell or cells, or a bodily fluid. More preferably, the sample comprises blood or a blood product or component. Most preferably, the sample comprises whole blood or blood plasma. Generally, the sample is from a mammal, such as a human. The term “analyte” is not particularly limiting. Suitable analytes may be any type of biomolecule which it is desired to detect in a sample. For example, the analyte may be a protein, peptide, carbohydrate, lipid, DNA or RNA, or whole cell, virus or bacterium. In particular, the analyte may be an antigen, a viral protein, a bacterial protein, an antibody, a specific DNA and/or RNA sequence, or specific cell type. In a specific embodiment of the present invention the analyte is related to the diagnosis and treatment (including the determination of theranostic information) of Hepatitis C. The method also extends to other human viruses such as HIV, cancer biomarkers and cells, cardiac markers, and markers for bacterial infections and any disease indications where multi-analyte information is important.

The term “sample” is not especially limiting and refers to any specimen in which an analyte may be present. In particular, as already mentioned, the sample may be whole blood, urine or other bodily fluid, or a crude lysate of solid tissue or cells. The sample may be subjected to processing steps before it is used in the present method.

The recognition agents referred to in the methods of the present invention are not especially limited. The particles may be coated with the recognition agent. The nature of the recognition agent is not especially limited, provided that it allows the particle to bind specifically to a target analyte. The recognition agent may be an antibody, specific for an antigen which may itself be the target analyte, or may be an antigen present on the surface of the target analyte. Alternatively, where the target analyte is a polynucleotide the recognition agent may be a polynucleotide sequence complementary to a section of the sequence of the analyte. In a further embodiment the recognition agent may be a lectin where the analyte is a carbohydrate. Recognition agents may also include those in the following systems: aptamer-polynucleotide; receptor-ligand; PNA-polynucleotide; and cell surface antigen-virus antigen. Where there are two or more analytes under investigation, an antibody specific for each analyte may be employed, to ensure that one particle type attaches to one analyte and a different particle type attaches to another analyte. In this manner, a plurality of analytes can be processed in the same sample.

In the present methods the particle that is buoyant in the fluid is not especially limited. Buoyant particles suitable for use in the present invention are, also commercially available. In particular the buoyant particle may be a hollow glass bead, such as those obtainable from Microsphere Technology Ltd, or any suppliers of buoyant particles for microfluidic applications.

Magnetic particles suitable for use in the present invention are well known in the art. In particular, magnetic beads are commercially available in a variety of sizes. In one embodiment the beads are super-paramagnetic beads. Such beads are preferred because regular magnetic beads tend to clump, when the magnetic field is not present which makes it difficult to wash and move them. Super-paramagnetic beads are only magnetic in a magnetic field, and do not suffer from clumping when the field is switched off. Thus, preferably the particles do not have any remnant magnetism.

The particles may also comprise a label to aid with their detection. The label may facilitate enzymatic, electrochemical (e.g. impedance), optical (e.g. fluorescence) or other detection methods.

The detection element for detecting an analyte may comprise any detection element, provided that the element is suitable for detecting the analyte under investigation. Preferably, the element comprises one or more of a biosensor array, an electrochemical biosensor element, and an optical biosensor element.

In a further preferred embodiment of this method, in one or more detecting conduits, an analyte may be concentrated according to a concentrating method as described above.

The invention also provides a method for detecting one or more analytes, which method comprises:
(a) separating an analyte, according to a method as defined above; and
(b) detecting the one or more analytes.

Further provided is a method of determining the presence of a pathogen in a sample from a subject, or determining the genotype of a subject from a sample, which method comprises:
detecting the absence or the presence and/or the quantity of the pathogen, or detecting the absence or the presence and/or the quantity of a protein, a polypeptide or a nucleic acid characteristic of the genotype, in the sample according to a method as defined above.

A particularly preferred example of this method is a method of detecting the presence of a pathogen in a subject, or detecting the presence of a genotype in a subject, which method comprises:
(a) obtaining a sample from the subject;
(b) detecting the absence or the presence and/or quantity of the pathogen, or detecting the absence presence and/or quantity of a protein, a polypeptide or a nucleic acid characteristic of the genotype, in the sample according to a method as defined above; and
(c) making a diagnosis of the subject, or determining the absence or presence of the genotype, based on the absence or the presence and/or quantity of the pathogen, or based on the absence or the presence and/or quantity of the polypeptide or nucleic acid characteristic of the genotype.

In the methods of the invention, the pathogen is typically selected from a bacterium and a virus, or wherein the polypeptide is selected from a protein or a protein fragment, or the nucleic acid is selected from DNA and RNA. More preferably, the pathogen is an HCV HBV, HAV, HIV, or Herpes simplex virus. Typically, the subject is a mammal, such as a human.

Still further provided is an apparatus for separating two or more analytes in a fluid, which apparatus comprises:
(a) a binding zone;
(b) two or more detecting zones;
(c) a separating conduit connecting the binding zone to the two or more detecting zones, each detecting zone comprising one or more detecting elements;
(d) a transporter for transporting the analyte through the separating conduit from the binding zone to the two or more detecting zones; and
(e) optionally, a concentrating zone in the vicinity of one or more of the detection elements.

As has been emphasised above, in all embodiments of the present invention, it is particularly preferred that the separating conduit is a microfluidic separating conduit and the functional conduits are microfluidic functional conduits.

The apparatus of the invention is typically a flow cell type apparatus. In the apparatus of the present invention, the transporter generally comprises a pump for pumping the fluid from the binding zone to the detection element. Typically, but not exclusively, the detecting element comprises a biosensor or a microarray.

The invention will now be described by way of example only, with reference to the following specific embodiments.

### EXAMPLES

Protocol for samples that are to be tested for HCV (this protocol may also be applied to HBV, HAV, HIV, or Herpes simplex, and also generally to other pathogens isolatable from specific bodily fluids)

Nature of the Sample

Typically the sample is whole blood, serum, plasma, cell lysate or extraction (such as B cells or hepatocytes), or urine. The sample may be conditioned to have a certain buffer composition, depending on the sample-type and its specific nature.

Bead Preparation

1 μg biotinylated antibody (other recognition agents, such as, oligonucleotides, PCR fragments, aptamers, PNA, lectins, antibody fragments, recombinant or purified receptors, and proteins may be employed as desired) to HCV E1 protein in 100 μl Phosphate Buffered Saline (PBS) is coupled to 300 μl buoyant (MST technologies) and magnetic (Dynal, Invitrogen) beads at 20×10⁶ beads/ml that have been coated with streptavidin by the manufacturer. The high affinity of biotin for streptavidin (dissociation constant [K_{D}]˜10⁻¹⁴M) ensures a successful reaction and allows the antibodies to coat the surface of the beads. The reaction is washed of excess uncoupled antibody by centrifuging the beads for 5 min at 14,000 rpm, discarding the supernatant and replacing with fresh PBS. This wash step is repeated twice.

Binding Step

The sample with a volume of 1-5 ml is incubated for several minutes with the beads that are coupled with antibodies that have been raised to HCV. This can also be achieved online, by flowing the sample at a rate of 0.1 to 5 ml/min over the beads in a chamber that allows retention of the sample (fritting material or filter) but permits the flow of solutions (preferred method). Through the same channel wash solution is passed after the sample, in a volume of 3 to 5 times the volume of the sample, to eliminate non-specific binding. This wash solution may contain detergents such as Triton X, Tween 20 or Nonidet P40 at concentrations of 0.01 to 1% that reduce the non-specific binding that can be observed in antibody-antigen interactions.

Flow Through to Sorting Mechanism or Detection Area

A valve on the microfluidic system is opened to allow the flow through of particles towards the sorting or biosensing area. Using low flow rates (0.01 to 1 ml/min) the beads are flowed through the system. During the flow step, depending on the geometry of the channels and the buoyancy of the beads, the particles that have bound the relevant entities are sorted into the relevant channels for detection and/or separation. If the mechanism is purely used to separate the beads, they are taken to a collection chamber where further processing,, if that is required, can take place.

If the beads are taken to a detection point or biosensor, they are flowed past it again at a low flow rate. The biosensor will be equipped with antibodies raised against another epitope of the virus, such as the E2 protein found on the envelope, or another epitope of the E1 envelope protein. Once bound, the beads that have not bound any biosensor recognition sites are flushed away using a wash solution, similar to that mentioned above.

Detection

If the beads are fluorescent they can be detected and counted immediately using a microscope or CCD camera. If the beads are not fluorescent a secondary antibody, raised to the primary antibody used on the bead, tagged with a fluorescent molecule or an enzyme capable of generating a chemiluminescent signal (such as Horse radish peroxidase-HRP) can be used (impedence methods, or enzymatic electrochemical detection methods may also be employed). This is flowed at a concentration of approximately 0.5 μg/ml over the bead complex. It is important that the secondary antibody does not cross-react or recognise the biosensor recognising entity. Detection is achieved by measuring the fluorescence emitted by the reaction using a microscope or a CCD camera.

1. A method for separating two or more analytes in a fluid, which method comprises:
(a) binding each different analyte to a different functional particle in one or more binding zones, to produce two or more bound analytes;
(b) allowing the bound analytes to move through a separating conduit to two or more separate functional zones;
wherein, each different functional particle has, or can be controlled to have, a different function in the fluid as compared with the other functional particles; and wherein the separating conduit separates into two or more functional conduits, such that the separating conduit serves to separate the bound analytes into the separate functional conduits by means of the different functions of the different functional particles.

2. The method according to claim 1, wherein the separating conduit is a microfluidic separating conduit and the functional conduits are microfluidic functional conduits.

3. The method according to claim 1, wherein the functional particle, or each different functional particle, is attached to a recognition agent that is specific for the analyte.

4. The method according to claim 3, wherein each functional particle is attached to a single recognition agent, or each functional particle is attached to all of the different recognition agents.

5. The method according to claim 4, wherein one or more of the functional conduits comprises a detection element.

6. The method according to claim 1, wherein the functional particle, or each different functional particle, is selected from:
(a) particles that are buoyant in the fluid;
(b) magnetic particles whose buoyancy can be controlled by the application of a magnetic field or whose buoyancy is neutral and whose attraction to the magnetic field can be controlled; and
(c) particles that are more dense than the fluid.

7. The method according to claim 1, wherein one or more of the recognition agents comprise an antibody.

8. The method according to claim 1, wherein the functional particle comprises a hollow glass bead that is buoyant in the fluid.

9. The method according to claim 1, wherein the fluid comprises a sample containing the analyte.

10. The method according to claim 9, wherein the sample comprises a lysate of solid tissue, a lysate of cells, a bodily fluid, blood or a blood product.

11. The method according to claim 10, wherein the sample comprises whole blood or blood plasma.

12. The method according to claim 9 wherein the sample is from a mammal.

13. The method according to claim 12 wherein the sample is from a human.

14. The method according to claim 1, wherein, the detection element for detecting an analyte comprises one or more of a biosensor array, an electrochemical biosensor element, and an optical biosensor element.

15. The method according to claim 1, wherein the analyte is selected from a biological molecule, a virus or virus component, and a cell or a cell component.

16. The method according to claim 15, wherein the analyte comprises a protein, a polypeptide, DNA and/or RNA.

17. A method for detecting one or more analytes, which method comprises:
(a) separating one or more analytes according to a method as defined in claim 1; and
(b) detecting the one or more analytes.

18. A method of determining the presence of a pathogen in a sample from a subject, or determining the genotype of a subject from a sample, which method comprises:
(a) separating one or more selected from a pathogen, protein, polypeptide, nucleic acid and any combination thereof according to a method as defined in claim 1; and
(b) detecting the absence or the presence and/or the quantity of the pathogen, or detecting the absence or the presence and/or the quantity of a protein a polypeptide or a nucleic acid characteristic of the genotype, in the sample.

19. The method according to claim 18, wherein the pathogen is selected from a bacterium and a virus, or wherein the polypeptide is selected from a protein or a protein fragment, or the nucleic acid is selected from DNA and RNA.

20. The method according to claim 19, wherein the pathogen is an HCV, HIV, or herpes virus.

21. The method according to claim 18 wherein the subject is a mammal.

22. The method according to claim 21 wherein the subject is human.

23. An apparatus for separating two or more analytes in a fluid, which apparatus comprises:
(a) a binding zone;
(b) two or more functional conduits;
(c) a separating conduit connecting the binding zone to the two or more functional conduits;
(d) a transporter for transporting the analyte through the separating conduit from the binding zone to the two or more functional conduits; and
(e) optionally one or more concentrating zones in connection with at least one of the functional conduits.

24. The apparatus according to claim 23, wherein the separating conduit is a microfluidic separating conduit and the functional conduits are microfluidic functional conduits.

25. The apparatus according to claim 23, further comprising at least one detecting element in at least one of the functional conduits.

26. The apparatus according to claim 25, comprising one or more detecting elements above one or more concentrating zones.

27. The apparatus according to claim 23, wherein the transporter comprises a pump for pumping the fluid from the binding zone.

28. The apparatus according to claim 23, wherein the detecting element is a biosensor or a microarray.
